(19) United States
(12) Patent Application Publication
(10) Pub. No.: US 2017/0253917 A1
(43) Pub. Date: Sep. 7, 2017

(54) METHODS AND SYSTEMS FOR DETERMINING ANTIBIOTIC SUSCEPTIBILITY

(71) Applicant: OpGen, Inc., Gaithersburg, MD (US)

(72) Inventors: George Terrance WALKER, Chevy Chase, MD (US); Tony ROCKWEILER, Arlington, VA (US)

(21) Appl. No.: 15/452,566

(22) Filed: Mar. 7, 2017

Related U.S. Application Data
(60) Provisional application No. 62/304,807, filed on Mar. 7, 2016.
(60) Provisional application No. 62/305,247, filed on Mar. 8, 2016.

(51) Int. Cl. C12Q 1/68; G06F 19/22; A61K 31/496; G06F 19/18; A61K 31/407; A61K 31/5383; A61K 31/546; G06F 19/28

(52) U.S. Cl. CPC C12Q 1/689; A61K 31/546; G06F 19/22; G06F 19/28; G06F 19/18; A61K 31/407; A61K 31/5383; A61K 31/496; C12Q 2600/158; G06F 19/325

(57) ABSTRACT

The present invention provides methods, systems, and kits for determining an appropriate therapeutic regimen for treating an infection caused by antibiotic resistant bacteria.

### CROSS REFERENCE TO RELATED APPLICATIONS

This application claims the benefit of, and priority to, provisional application U.S. 62/304,807, filed Mar. 7, 2016, and provisional application U.S. 62/305,247, filed Mar. 8, 2016, the contents of which are herein incorporated by reference in their entireties.

### FIELD OF THE INVENTION

The invention relates generally to the rapid determination of the antibiotic susceptibility of a microorganism, such as, an infectious microorganism in a biological sample using genetic information. Methods of the invention may be applied to the rapid identification, typing, antibiotic susceptibility determination, and/or antibiotic minimum inhibitory concentration (MIC) determination for any infectious microorganism, such as a Gram positive bacteria or a Gram negative bacteria.

### BACKGROUND OF THE INVENTION

Microorganism infections, such as bacteremia, sepsis, and pneumonia, are frequently associated with multi-drug-resistant organisms (MDRO). According to the Centers for Disease Control and Prevention, MDROs are defined as microorganisms that are resistant to three or more classes of antimicrobial agents. Rapid and accurate methods of microorganism identification and drug susceptibility testing are essential for disease diagnosis, treatment of infection, and to trace disease outbreaks associated with microbial infections.

Traditional methods of microorganism identification involve conventional microbiological procedures (i.e., isolating a pure colony of the microorganism in question and then culturing that isolate on solid medium or in liquid phase) followed by analysis of the biochemical and/or phenotypic characteristics of the organism (i.e., gram staining and/or DNA analysis). Traditional methods of drug susceptibility testing typically require the isolation of a pure colony of the microorganism in question and then analysis of the growth of that isolate using a broth dilution or agar diffusion assay.

The broth dilution method involves inoculating a pure isolate of the microorganism in question into a growth medium (typically, Mueller Hinton broth) containing a series of predetermined concentrations of the particular antibiotic for which a minimum inhibitory concentration (MIC), or an MIC-like measurement, is to be determined. The inoculated medium is incubated for 18-24 hours and observed for visible growth, as measured by turbidity, pellet size, and/or release of the chromogenic or fluorogenic moiety. The lowest antibiotic concentration that completely inhibits visible growth of the isolated organism is recorded as the MIC.

The agar diffusion assay involves the placement of an antibiotic containing disc or an antibiotic gradient strip on the surface of an agar medium (typically, a Mueller Hinton agar plate) that has been inoculated with a pure isolate of the microorganism in question. The plates are incubated for 18-24 hours, during which time the antibiotic substance diffuses away from the disc or strip, such that the effective concentration of antibiotic varies as a function of the radius from the disc or strip. The diameter of the resulting area of no growth and/or no color (i.e., the zone of inhibition) around the disc or strip, if any, is directly proportional to the MIC.

Current FDA-approved methods for antibiotic susceptibility testing require inoculation of around 10⁵ CFU/mL microorganisms. Because clinical samples generally contain substantially less than 10⁵ CFU/mL, it is difficult to apply FDA-approved tests directly to clinical specimens. Typically, clinical samples are inoculated into culture medium and grown until the number of microorganisms reaches about 10⁸ CFU/mL. Usually, the processes of microorganism identification and antibiotic susceptibility testing require 48 to 72 hours to be completed, during which time the microorganism continues to spread in the patient and in the environment.

Shortening the time necessary to identify the infectious microorganism and select an effective antibiotic regimen could significantly decrease morbidity and mortality rates, prevent epidemic outbreaks, and reduce the cost of treating patients with aggressive microorganism infections.

Accordingly, a primary object of the invention is to provide a method for rapid microorganism detection and drug susceptibility screening.

### SUMMARY OF THE INVENTION

One aspect of the present invention is a method for predicting phenotypic antibiotic resistance of a pathogenic bacteria. The method includes steps of detecting in the bacteria the presence or absence of at least one antibiotic resistance gene to produce an infection source profile and comparing the infection source profile to a control profile thereby predicting the phenotypic antibiotic resistance of the bacteria. In embodiments, the bacteria may be obtained from a biological sample from a subject having or suspected of having a pathogenic bacterial infection or the bacteria may be collected from the environment.

One aspect of the present invention is a method for determining the minimal inhibitory concentration (MIC) of an antibiotic that treats a bacterial infection in a subject. The method includes steps of obtaining a biological sample (e.g., comprising pathogenic bacteria) from the subject, detecting in the biological sample the presence or absence of at least one antibiotic resistance gene to produce an infection source profile, and comparing the infection source profile to a control profile thereby identifying the MIC of the antibiotic that treats the bacterial infection. The method may further comprise choosing and administering the antibiotic to the subject at a dose based on the MIC. In embodiments, the subject has or is suspected of having a bacterial infection. In embodiments, the control profile is a database.

Any of the above aspects or embodiments, the biological sample may be an anal swab, a rectal swab, a skin swab, a nasal swab, a wound swab, stool, blood, plasma, serum, urine, sputum, respiratory lavage, cerebrospinal fluid, or a bacterial culture.

An additional aspect of the present invention is a method for determining the minimal inhibitory concentration (MIC) of an antibiotic for a bacterial isolate. The method includes steps of detecting in the bacterial isolate the presence or absence of at least one antibiotic resistance gene to produce an infection source profile and comparing the infection source profile to a control profile thereby identifying the MIC of the antibiotic for the bacterial isolate. In embodiments, the bacterial isolate may be obtained from a subject having or suspected of having a bacterial infection or the bacterial isolate may be collected from the environment.

Yet another aspect of the present invention is a method for determining whether an infection source will be susceptible to an antibiotic comprising. The method includes steps of obtaining a sample comprising the infection source, detecting in the sample the presence or absence of an antibiotic resistance gene thereby determining an infection source profile, and comparing the infection source profile to a control profile thereby determining whether an infection source will be susceptible to an antibiotic. In embodiments, the sample may be obtained from a subject having or suspected of having a bacterial infection or the sample may be collected from the environment.

An aspect of the present invention is a method for generating a database that correlates a genetic profile with a minimal inhibitory concentration (MIC) of an antibiotic. The method compromises steps of obtaining a plurality of bacterial isolates of a bacterial species or a bacterial strain wherein the MIC of the antibiotic for each bacterial isolate in the plurality is known, determining a genetic profile for each bacterial isolate, wherein the genetic profile comprises the presence or absence of one or more antibiotic resistance genes, and associating each genetic profile for each isolate with its known MIC of the antibiotic, thereby generating a database that correlates a genetic profile with a MIC of the antibiotic. The present invention also includes the database generated by this method. Also included is a non-transient computer readable medium containing the database.

Another aspect of the present invention is a method for generating a database that correlates a genetic profile with susceptibility to an antibiotic. The method comprises steps of obtaining a plurality of bacterial isolates of a bacterial species or a bacterial strain wherein each bacterial isolate in the plurality has a known susceptibility to at least one antibiotic, determining a genetic profile for each isolate wherein the genetic profile comprises the presence or absence of one or more antibiotic resistance genes, and associating each genetic profile for each isolate with its known susceptibility to the at least one antibiotic, thereby generating a database that correlates a genetic profile with susceptibility to at least one antibiotic. The present invention also includes the database generated by this method. Also included is a non-transient computer readable medium containing the database.

An additional aspect of the present invention is a method for predicting phenotypic antibiotic resistance of a pathogenic bacteria. The method comprises steps of detecting in the bacteria the presence or absence of at least one antibiotic resistance gene to produce an infection source profile and comparing the infection source profile to a database of one of the previous two aspects, thereby predicting the phenotypic antibiotic resistance of the bacteria. In embodiments, the bacteria may be obtained from a subject having or suspected of having a pathogenic bacterial infection or the bacteria may be collected from the environment.

Yet another aspect of the present invention is a method of identifying the bacterial species or bacterial strain in a sample. The method comprises steps of detecting in the sample the presence or absence of at least one antibiotic resistance gene to produce a sample profile and comparing the sample profile to a control profile thereby identifying the bacterial strain in a sample. In embodiments, the sample may be obtained from a subject having or suspected of having a bacterial infection or the sample may be collected from the environment.

An aspect of the present invention is a method for predicting phenotypic antibiotic resistance of a pathogenic bacteria. The method comprises steps of assessing the expression of a plurality of antibiotic resistance genes in the bacteria and calculating a score from the expression the antibiotic resistance genes wherein the score indicates the phenotypic resistance of the bacteria. In embodiments, the bacteria may be obtained from a subject having or suspected of having a bacterial infection or the bacteria may be collected from the environment.

In any of the above aspects or embodiments, when a sample, bacteria, or bacterial isolate is obtained from the environment, the method may further comprise making a contact precautions recommendation, e.g., one or more of isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

In any of the above aspects or embodiments, the antibiotic resistance gene may be aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, or VIM-5.

In any of the above aspects or embodiments, the antibiotic may be Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, or Vancomycin.

In any of the above aspects or embodiments, the bacteria may be from the species Escherichia coli, Klebsiella pneumoniae, Enterobacter cloacae, Pseudomonas aeruginosa, Proteus mirabilis, Klebsiella oxytoca, Streptococcus pneumoniae, Staphylococcus aureus, Streptococcus anginosus, Streptococcus constellatus, Streptococcus salivarius, Enterobacter aerogenes, Serratia marcescens, Acinetobacter baumannii, Citrobacter freundii, Morganella morganii, Legionella pneumophila, Moraxella catarrhalis, Haemophilus influenzae, Haemophilus parainfluenzae, Mycoplasma pneumoniae, Chlamydophila pneumoniae, Clostridium species, or Bacteroides fragilis.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting. Other features and advantages of the invention will be apparent from and encompassed by the following detailed description and claims.

### BRIEF DESCRIPTION OF THE DRAWINGS

The above and further features will be more clearly appreciated from the following detailed description when taken in conjunction with the accompanying drawings.

FIG. 1 includes a decision tree for susceptibility to the antibiotic Cefepime. The decision tree includes positive/negative results for the antibiotic resistance genes KPC, CTX-M-1, CTX-M-9, VEB, and NDM.

FIG. 2 includes a decision tree for susceptibility to the antibiotic Levofloxacin. Levofloxacin minimum inhibitory concentration (MIC) values are based on genotypes for three genes.

FIG. 3 includes a comparison of measured minimum inhibitory concentration (MIC) values from phenotypic AST to predicted MIC values for isolates of Klebsiella. Cefepime minimum inhibitory concentration (MIC) values are based on genotypes for beta-lactamase genes.

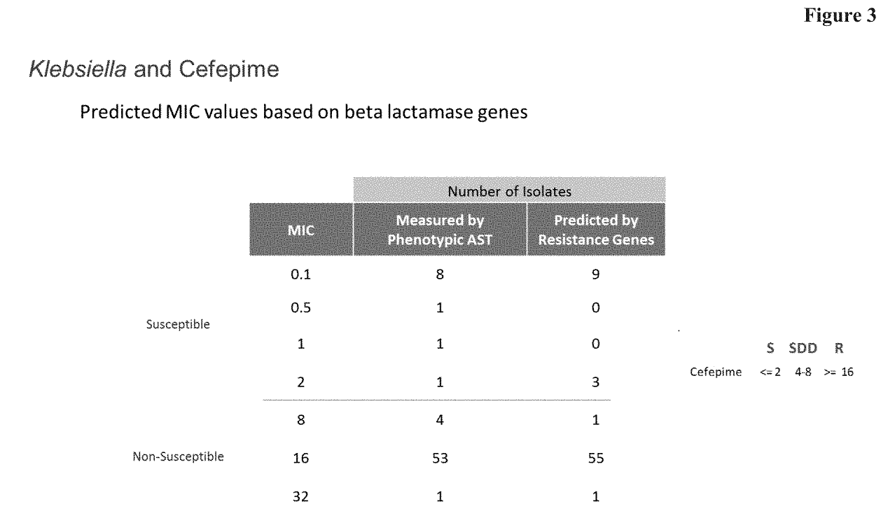

FIG. 4 includes a comparison of resistance genes in Klebsiella that predict susceptibility to the antibiotic Cefepime.

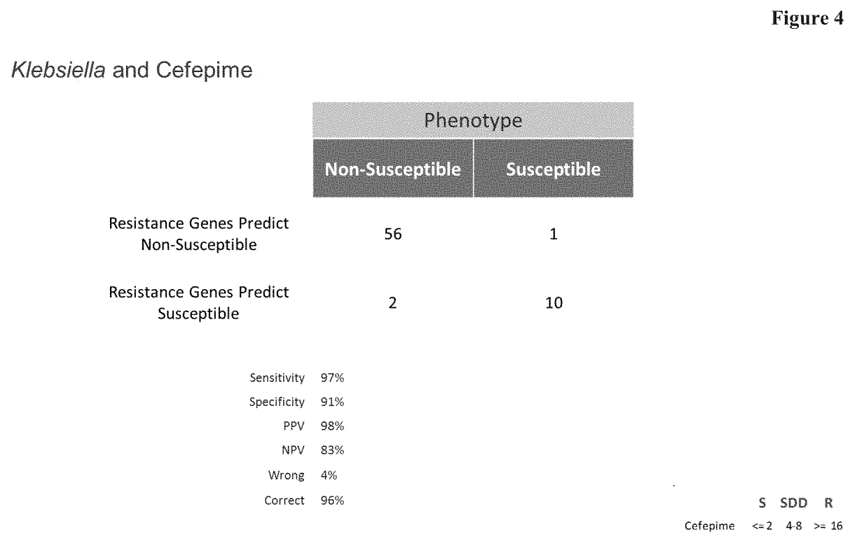

FIG. 5 includes predicted non-susceptibility of Klebsiella and E. coli to the antibiotics Ceftazidime, Cefepime, Etrapenem, Meropenem, and Imipenem.

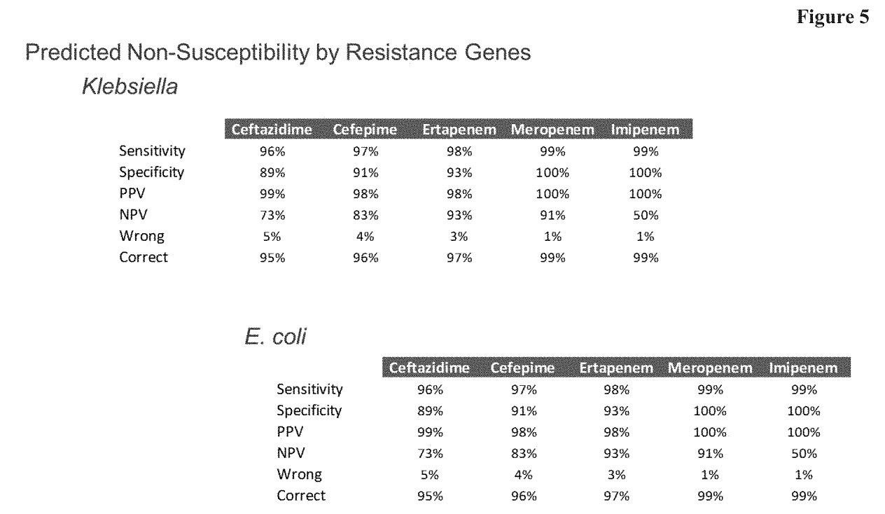

FIG. 6 includes a comparison of measured minimum inhibitory concentration (MIC) values from phenotypic AST to predicted MIC values for isolates of Pseudomonas aeruginosa. Levofloxacin predicted minimum inhibitory concentration (MIC) values are based on mutation of P. aeruginosa DNA gyrase.

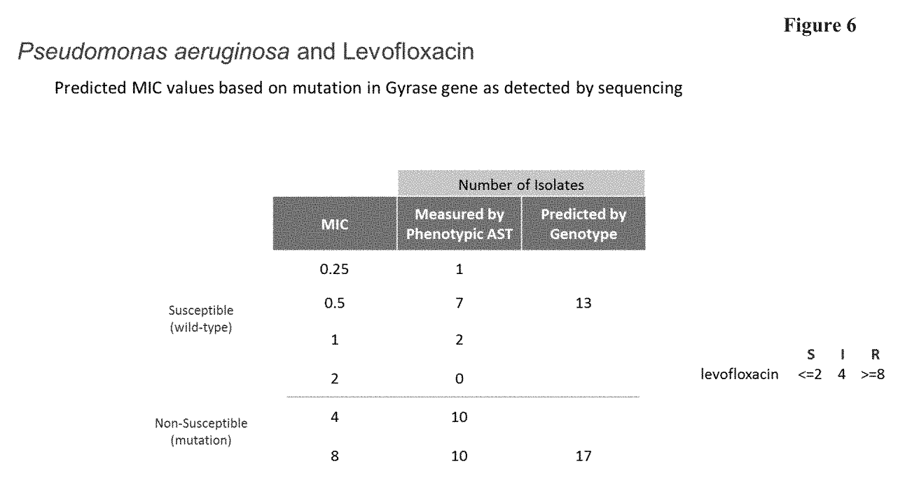

FIG. 7 includes a comparison of gyrase genotypes in P. aeruginosa that predict susceptibility to the antibiotic Levofloxacin.

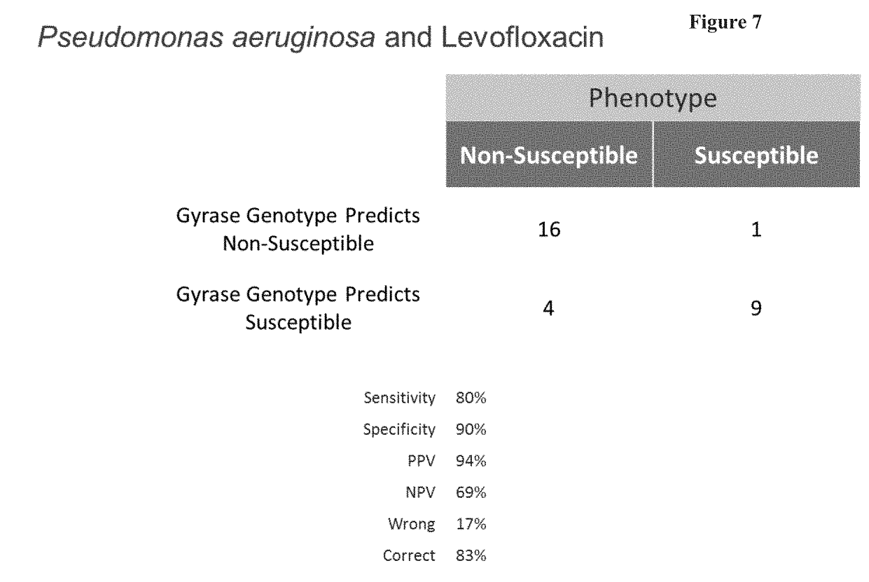

FIG. 8 includes predicted non-susceptibility of P. aeruginosa, E. coli, and Klebsiella pneumonia to Levofloxacin and Ciprofloxacin.

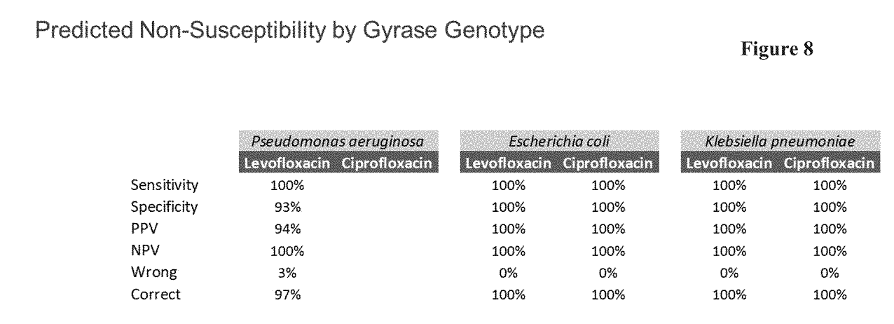

FIG. 9 includes individual heat maps for 30 of the 1496 E. coli isolates based on the presence of antibiotic resistance genes.

### DETAILED DESCRIPTION OF THE INVENTION

The present invention is based upon the surprising discovery that the minimal inhibitory concentration (MIC) value of an antibiotic for a bacterial can be determined by genotyping the bacteria. Specifically, by obtaining the genotype of the bacterial by detecting a set of antibiotic resistance genes and combining these results with phenotypic antibiotic susceptibility test (AST) results a predictive algorithm for susceptibility was created. The decision tree was used to evaluate antibiotic resistance gene results from the test set of bacterial isolates to predict MIC values that were compared with measured MIC values from phenotypic AST. Gene test results were able predict phenotypic AST with extremely high sensitivity and specificity.

Accordingly, the present invention provides systems and method for predicting phenotypic resistance based upon the bacteria genotype with respect to a set of antibiotic resistance genes. The systems and methods of the invention allows for the rapid determination of an appropriate therapeutic regimen for treating an infection. Importantly, the systems and methods of the invention provide a rapid (several days ahead of AST) method for determining antibiotic resistance of a bacterial infection or bacterial isolate, allowing for proper antibiotic selection. As such, the systems and methods of the invention improve patient management.

Additionally, the systems and methods of the invention allow for the creation of a database that allows phenotypic resistance to be determined by the bacteria's genotype. The database is useful for cataloging and tracking resistance in a digital manner.

The methods disclosed herein identify, in a biological sample, a genetic profile of an infection source, i.e., infection source profile. The infection source is one bacterial species or strain or a plurality of bacterial species or strains that produces an infection in a subject. The infection source profile includes the set of one or more antibiotic resistance genes detected in the biological sample or an extract of the biological sample. The infection source profile is compared to a control profile, e.g., a database, which includes information associating antibiotic resistance genes with susceptibility or resistance to specific antibiotics. The database further includes information regarding the minimal inhibitory concentration (MIC) of an antibiotic that treats a bacterial infection in a subject. The database further includes genetic profiles for known bacterial species and strains; thus, the database may be used to determine the species or strain of infection source based upon its infection source profile. Together, these methods allow a health care professional to determine an appropriate therapeutic regimen, including one or more antibiotics, for treating an infection due to one or more antibiotic resistant bacteria.

### Definitions

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, In some embodiments, to a only (optionally including elements other than B); in another embodiment, to B only (optionally including elements other than a); in yet another embodiment, to both a and B (optionally including other elements).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, In some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “plurality” is meant more than one, i.e., 2, 3, 4, 5, 6, 7, 8, 9, 10, 100, 1,000, 10,000, 100,000 or more and any number in between.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, the terms “about” and “approximately” are interchangeable, and should generally be understood to refer to a range of numbers around a given number, as well as to all numbers in a recited range of numbers (e.g., “about 5 to 15” means “about 5 to about 15” unless otherwise stated). “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Moreover, all numerical ranges herein should be understood to include each whole integer within the range.

As used herein, the term “e.g.” is used merely by way of example, without limitation intended, and should not be construed as referring only those items explicitly enumerated in the specification. As used herein, the term “antibiotic susceptibility testing” refers to any test or assay for evaluating microorganisms for their susceptibility to antibiotics of interest. An antibiotic susceptibility test may be used to determine the clinical efficacy of an antibiotic for treating infection caused by a microorganism.

As used herein, the terms “susceptible” and “antibiotic susceptibility” indicate that the growth of a microorganism is inhibited by the usually achievable concentrations of an antimicrobial agent when the recommended dosage is used.

As used herein, the terms “intermediate” and “intermediate susceptibility” indicate that at the minimum inhibitory concentration (MIC) of an antimicrobial agent, which approaches usually attainable blood and tissue levels, growth of a microorganism is higher than for susceptible microorganisms. Intermediate susceptibility indicates clinical efficacy in body sites where the antimicrobial agents are physiologically concentrated or when a higher than normal dosage can be used.

As used herein, the terms “resistant” and “antibiotic resistance” indicate that microorganism growth is not inhibited by the usually achievable concentrations of the agent with normal dosage schedules and clinical efficacy of the agent against the microorganism has not been shown in treatment studies. These terms also indicate situations in which the microorganisms exhibit specific microbial resistance mechanisms.

As used herein, an “infection source” is one microbe or a set of microbes, e.g., bacteria, which infect a subject. The infection source may be a single species or strain of bacterium. Alternately, an infection source may include two or more bacterial species or bacterial strains, e.g., at least 3, 4, 5, 10, 20, 50, and 100, or any number in between.

As used herein, the term “infection” or “bacterial infection” is meant to include any infectious agent of bacterial origin. The bacterial infection may be the result of Gram-positive, Gram-negative bacteria or atypical bacteria. In embodiments, the infectious agent is a pathogenic bacteria. Non-limiting examples of pathogenic bacteria include: Escherichia coli, Klebsiella pneumoniae, Enterobacter cloacae, Pseudomonas aeruginosa, Proteus mirabilis, Klebsiella oxytoca, Streptococcus pneumoniae, Staphylococcus aureus, Streptococcus anginosus, Streptococcus constellatus, Streptococcus salivarius, Enterobacter aerogenes, Serratia marcescens, Acinetobacter baumannii, Citrobacter freundii, Morganella morganii, Legionella pneumophila, Moraxella catarrhalis, Haemophilus influenzae, Haemophilus parainfluenzae, Mycoplasma pneumoniae, Chlamydophila pneumoniae, Clostridiumspecies, or Bacteroides fragilis.

An antimicrobial is a drug or compound or chemical used in the treatment or prevention of a microbial infection. They may either kill or inhibit the growth of the microbe. Antibiotics or antibacterials are a type of antimicrobial used in the treatment or prevention of bacterial infection. They may either kill or inhibit the growth of bacteria. Antibiotics include for example. penicillins, cephalosporins, carbapenems, aminoglycosides, fluoroquinolones, tetracyclines and/or trimethoprim/sulfamethoxazole. Non-limiting examples of antibiotics include: Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, and Vancomycin.

An antibiotic resistance gene provides a bacteria comprising said gene resistance to a specific antibiotic. Many antibiotic resistance genes are known in the art. Non-limiting examples of antibiotic resistance genes include: aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, and VIM-5. An infection source may comprise one antibiotic resistance gene or two or more resistance genes, e.g., 3 or more, 4 or more, 5 or more, 10 or more, 20 or more, and 100 or more or any number in between.

A bacterium that lacks a particular antibiotic resistance gene may be susceptible to one or more specific antibiotics.

As used herein, an “infection source profile” is, at least, an identified antibiotic resistance gene that a bacterium, bacterial isolate, or biological sample comprises or a set of identified antibiotic resistance genes that a bacterium, bacterial isolate, or biological sample comprises.

As used herein, a “control profile” is, at least, one identified antibiotic resistance gene that is known to confer resistance to a specific antibiotic or a plurality of specific antibiotics; a “control profile” may also be, at least, a set of identified antibiotic resistance genes that are known to confer resistance to a specific antibiotic or a plurality of antibiotics. The control profile may be a database, e.g., a digital database that may be recorded on a non-transient computer readable medium. The control profile allows a user to associate an infection source profile with an antibiotic or a plurality of specific antibiotics to which the bacterium, bacterial isolate, or biological sample is predicted to be sensitive or resistant.

The database may include information regarding one or more specific antibiotics to which a known bacteria, a known bacterial isolate, or a known biological sample is resistant or sensitive to.

The database may further include information regarding the MIC for one or more specific antibiotics to which the known bacteria, known bacterial isolate, or known biological sample is sensitive. The database may further include information regarding the MIC for one or more specific antibiotics for a particular control profile.

The database may allow prediction of antibiotic resistance or sensitivity of unknown bacteria, bacterial isolate, or biological sample based upon its infection source profile. Further, the database may allow identification of a bacterial species and/or bacterial strain based upon its infection source profile.

The database, which associates a “control profile” with susceptibility or resistance to at least one antibiotic, can be generated using any algorithm available to a skilled artisan. Commercial, shareware, and freeware algorithms may be used to generate a database, e.g., RapidMiner Studio.

As used herein, the terms “treat,” treating,” “treatment,” and the like refer to reducing or ameliorating a disease, infection, disorder, or condition and/or a symptom associated therewith. It will be appreciated that, although not precluded, treating a disease, infection, disorder, or condition does not require that the disease, infection, disorder, or condition or symptoms associated therewith be completely eliminated. Treating may include a health care professional or diagnostic scientist making a recommendation to a subject for a desired course of action or treatment regimen, e.g., a prescription. As used herein, a “method of treating” includes a method of managing, and when used in connection with the biological organism or infection, may include the amelioration, elimination, reduction, prevention, and/or other relief from a detrimental effect of a biological organism.

As used herein, the terms “prevent,” “preventing,” “prevention,” “prophylactic treatment” and the like refer to reducing the probability of developing a disease, infection, disorder, or condition in a subject, who does not have, but is at risk of or susceptible to developing a disease, infection, disorder, or condition.

Methods of treating or preventing may include administering to a subject a therapeutic regimen comprising one or more antibiotics. Also considered by the terms “treating” or “preventing” include providing to the subject a recommendation for a therapeutic regimen comprising at least one antibiotic, e.g., a prescription for one or more antibiotics.

As used herein, the terms “drug”, “medication”, “therapeutic”, “active agent”, “therapeutic compound”, “composition”, or “compound” are used interchangeably and refer to any chemical entity, pharmaceutical, drug, biological, botanical, and the like that can be used to treat or prevent a disease, infection, disorder, or condition of bodily function, e.g., a bacterial infection. A drug may comprise both known and potentially therapeutic compounds. A drug may be determined to be therapeutic by screening using the screening known to those having ordinary skill in the art. A “known therapeutic compound”, “drug”, or “medication” refers to a therapeutic compound that has been shown (e.g., through animal trials or prior experience with administration to humans) to be effective in such treatment. A “therapeutic regimen” relates to a treatment comprising a “drug”, “medication”, “therapeutic”, “active agent”, “therapeutic compound”, “composition”, or “compound” as disclosed herein and/or a treatment comprising behavioral modification by the subject and/or a treatment comprising a surgical means. In preferred embodiments, the drug is an antibiotic that kills or inhibits the growth of a bacteria or plurality of bacteria.

“Accuracy” refers to the degree of conformity of a measured or calculated quantity (a test reported value) to its actual (or true) value. Clinical accuracy relates to the proportion of true outcomes (true positives (TP) or true negatives (TN) versus misclassified outcomes (false positives (FP) or false negatives (FN)), and may be stated as a sensitivity, specificity, positive predictive values (PPV) or negative predictive values (NPV), or as a likelihood, odds ratio, among other measures.

Using such statistics, an “acceptable degree of diagnostic accuracy”, is herein defined as a test or assay in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

A “Clinical indicator” is any physiological datum used alone or in conjunction with other data in evaluating the physiological condition of a collection of cells or of an organism. This term includes pre-clinical indicators.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. In panel and combination construction, of particular interest are structural and synactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful as forward selection, backwards selection, or stepwise selection, complete enumeration of all potential panels of a given size, genetic algorithms, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art. A “health economic utility function” is a formula that is derived from a combination of the expected probability of a range of clinical outcomes in an idealized applicable patient population, both before and after the introduction of a diagnostic or therapeutic intervention into the standard of care. It encompasses estimates of the accuracy, effectiveness and performance characteristics of such intervention, and a cost and/or value measurement (a utility) associated with each outcome, which may be derived from actual health system costs of care (services, supplies, devices and drugs, etc.) and/or as an estimated acceptable value per quality adjusted life year (QALY) resulting in each outcome. The sum, across all predicted outcomes, of the product of the predicted population size for an outcome multiplied by the respective outcomes expected utility is the total health economic utility of a given standard of care. The difference between (i) the total health economic utility calculated for the standard of care with the intervention versus (ii) the total health economic utility for the standard of care without the intervention results in an overall measure of the health economic cost or value of the intervention. This may itself be divided amongst the entire patient group being analyzed (or solely amongst the intervention group) to arrive at a cost per unit intervention, and to guide such decisions as market positioning, pricing, and assumptions of health system acceptance. Such health economic utility functions are commonly used to compare the cost-effectiveness of the intervention, but may also be transformed to estimate the acceptable value per QALY the health care system is willing to pay, or the acceptable cost-effective clinical performance characteristics required of a new intervention.

For diagnostic (or prognostic) interventions of the invention, as each outcome (which in a disease classifying diagnostic test may be a TP, FP, TN, or FN) bears a different cost, a health economic utility function may preferentially favor sensitivity over specificity, or PPV over NPV based on the clinical situation and individual outcome costs and value, and thus provides another measure of health economic performance and value which may be different from more direct clinical or analytical performance measures. These different measurements and relative trade-offs generally will converge only in the case of a perfect test, with zero error rate (a.k.a., zero predicted subject outcome misclassifications or FP and FN), which all performance measures will favor over imperfection, but to differing degrees.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's non-analyte clinical parameters.

“Negative predictive value” or “NPV” is calculated by TN/(TN+FN) or the true negative fraction of all negative test results. It also is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al, “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428. An alternative approach using likelihood functions, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

Finally, hazard ratios and absolute and relative risk ratios within subject cohorts defined by a test are a further measurement of clinical accuracy and utility. Multiple methods are frequently used to defining abnormal or disease values, including reference limits, discrimination limits, and risk thresholds.

“Analytical accuracy” refers to the reproducibility and predictability of the measurement process itself, and may be summarized in such measurements as coefficients of variation, and tests of concordance and calibration of the same samples or controls with different times, users, equipment and/or reagents. These and other considerations in evaluating new biomarkers are also summarized in Vasan, 2006.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Positive predictive value” or “PPV” is calculated by TP/(TP+FP) or the true positive fraction of all positive test results. It is inherently impacted by the prevalence of the disease and pre-test probability of the population intended to be tested.

“Sensitivity” of an assay is calculated by TP/(TP+FN) or the true positive fraction of disease subjects.

“Specificity” of an assay is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is considered highly significant at a p-value of 0.05 or less. Preferably, the p-value is 0.04, 0.03, 0.02, 0.01, 0.005, 0.001 or less.

“TN” is true negative, which for a disease state test means classifying a non-disease or normal subject correctly.

“TP” is true positive, which for a disease state test means correctly classifying a disease subject.

As used herein, “subject” (also interchangeably referred to as “host” or “patient”) refers to any host that may serve as a source of one or more of the biological samples or specimens as discussed herein and/or has or is suspected of having a bacterial infection. In certain aspects, the subject will be a vertebrate animal, which is intended to denote any animal species (and preferably, a mammalian species such as a human being). In certain embodiments, a subject refers to any animal, including but not limited to, human and non-human primates, avians, reptiles, amphibians, bovines, canines, caprines, cavities, corvines, epines, equines, felines, hircines, lapines, leporines, lupines, ovines, porcines, racines, vulpines, and the like, including, without limitation, domesticated livestock, herding or migratory animals or birds, exotics or zoological specimens, as well as companion animals, pets, and any animal under the care of a veterinary practitioner.

As used herein, “sample” includes anything containing or presumed to contain a substance of interest. It thus may be a composition of matter containing nucleic acid, protein, or another biomolecule of interest. The term “sample” may thus encompass a solution, cell, tissue, or population of one of more of the same that includes a population of nucleic acids (genomic DNA, cDNA, RNA, protein, and other cellular molecules). The terms “nucleic acid source,” “sample,” and “specimen” are used interchangeably herein in a broad sense, and are intended to encompass a variety of biological sources that contain nucleic acids, protein, one or more other biomolecules of interest, or any combination thereof. Exemplary biological samples include, but are not limited to, whole blood, plasma, serum, sputum, urine, stool, white blood cells, red blood cells, buffy coat, swabs (including, without limitation, buccal swabs, throat swabs, vaginal swabs, urethral swabs, cervical swabs, rectal swabs, lesion swabs, abscess swabs, nasopharyngeal swabs, and the like), urine, stool, sputum, tears, mucus, saliva, semen, vaginal fluids, lymphatic fluid, amniotic fluid, spinal or cerebrospinal fluid, peritoneal effusions, pleural effusions, exudates, punctates, epithelial smears, biopsies, bone marrow samples, fluids from cysts or abscesses, synovial fluid, vitreous or aqueous humor, eye washes or aspirates, bronchial or pulmonary lavage, lung aspirates, and organs and tissues, including but not limited to, liver, spleen, kidney, lung, intestine, brain, heart, muscle, pancreas, and the like, and any combination thereof. Tissue culture cells, including explanted material, primary cells, secondary cell lines, and the like, as well as isolates, lysates, homogenates, extracts, or materials obtained from any cells, are also within the meaning of the term “biological sample,” as used herein. The ordinary-skilled artisan will also appreciate that isolates, lysates, extracts, or materials obtained from any of the above exemplary biological samples are also within the scope of the invention.

The method involves extraction of bacterial nucleic acids from a biological sample from a subject or directly from a biological sample culture or culture isolate. Extraction can be accomplished by any known method in the art. Preferably, the extraction method both isolates and purifies the nucleic acid. By “purifies” is meant that the resulting extracted nucleic acid is substantially free of protein, cellular debris, and PCR inhibitors. Methods of extraction suitable for use in the present invention include, for example but not limited to Roche MagNAPure.

As used herein, a “bacteria isolate” is biological sample comprising a bacterium or a bacterial component (e.g., a nucleic acid). Alternately, a bacteria isolate may be a bacterium or a bacterial component isolated from the biological sample. Additionally, a bacteria isolate may be obtained from a bacterial culture.

As used herein, the phrases “isolated” or “biologically pure” may refer to material that is substantially, or essentially, free from components that normally accompany the material as it is found in its native state. Thus, isolated polynucleotides in accordance with the invention preferably do not contain materials normally associated with those polynucleotides in their natural, or in situ, environment.

The term “substantially free” or “essentially free,” as used herein, typically means that a composition contains less than about 10 weight percent, preferably less than about 5 weight percent, and more preferably less than about 1 weight percent of a compound. In a preferred embodiment, these terms refer to less than about 0.5 weight percent, more preferably less than about 0.1 weight percent or even less than about 0.01 weight percent. The terms encompass a composition being entirely free of a compound or other stated property, as well. With respect to degradation or deterioration, the term “substantial” may also refer to the above-noted weight percentages, such that preventing substantial degradation would refer to less than about 15 weight percent, less than about 10 weight percent, preferably less than about 5 weight percent, being lost to degradation. In other embodiments, these terms refer to mere percentages rather than weight percentages, such as with respect to the term “substantially non-pathogenic” where the term “substantially” refers to leaving less than about 10 percent, less than about 5 percent, of the pathogenic activity.

As used herein, “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snoRNA), small nuclear RNA (snRNA), and small temporal RNA (stRNA), and the like, and any combination thereof.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained, and also in the case of pathogens, optionally isolated away from, or purified free from total mammalian (preferably human) genomic DNA of the infected individual. Included within the term “DNA segment,” are DNA segments and smaller fragments of such segments, as well as recombinant vectors, including, for example, plasmids, cosmids, phage, viruses, and the like.

Similarly, the term “RNA segment” refers to an RNA molecule that has been isolated free of total cellular RNA of a particular species. Therefore, RNA segments obtained from a biological sample using one of the compositions disclosed herein, refers to one or more RNA segments (either of native or synthetic origin) that have been isolated away from, or purified free from, other RNAs. Included within the term “RNA segment,” are RNA segments and smaller fragments of such segments.

As used herein, the terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity may be determined by analyzing two or more sequences using algorithms and computer programs known in the art. Such methods may be used to assess whether a given sequence is identical or homologous to another selected sequence.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the phrase “substantially identical,” in the context of two nucleic acids refers to two or more sequences or subsequences that have at least about 90%, preferably 91%, most preferably about 92%, 93%, 94%, 95%, 96%, 97%, 98%, 98.5%, 99%, 99.1%, 99.2%, 99.3%, 99.4%, 99.5%, 99.6%, 99.7%, 99.8%, 99.9% or more nucleotide residue identity, when compared and aligned for maximum correspondence, as measured using a sequence comparison algorithm or by visual inspection. Such “substantially identical” sequences are typically considered “homologous,” without reference to actual ancestry.

As used herein, a “primer” or “primer sequence” may include any nucleic acid sequence or segment that selectively hybridizes to a complementary template nucleic acid strand (“target sequence”) and functions as an initiation point for the addition of nucleotides to replicate the template strand. Primer sequences of the present disclosure may be labeled or contain other modifications which allow the detection and/or analysis of amplification products. In addition to serving as initiators for polymerase-mediated duplication of target DNA sequences, primer sequences may also be used for the reverse transcription of template RNAs into corresponding DNAs.

As used herein, a “probe” or “probe sequence” may include any nucleic acid sequence or segment that selectively hybridizes to a complementary target nucleic acid or target nucleic acid strand (“target sequence”) and functions to identify said target sequence.

As used herein, a “target sequence” or “target nucleotide sequence” as used herein includes any nucleotide sequence to which one of the disclosed primer sequences hybridizes under conditions that allow an enzyme having polymerase activity to elongate the primer sequence, and thereby replicate the complementary strand.

The present invention also encompasses nucleic acid segments that are complementary, essentially complementary, and/or substantially complementary to at least one or more of the specific nucleotide sequences specifically set forth herein. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to one or more of the specific nucleic acid segments disclosed herein under relatively stringent conditions such as those described immediately above. Examples of nucleic acid segments are amplification (PCR) primers and (detection) probes.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorigenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernable from the first label. The use of multiplexing assays, particularly in the context of genetic amplification/detection protocols are well-known to those of ordinary skill in the molecular genetic arts.

In general, it is envisioned that one or more of the amplification primers and/or hybridization probes described herein will be useful both as reagents in solution hybridization (e.g., PCR methodologies and the like), and in embodiments employing “solid-phase” analytical protocols and such like.

Following collection of a biological sample, any method of nucleic acid extraction or separation from the sample may be performed, as would be known to one of ordinary skill in the art, including, but not limited to, the use of the standard phenol/chloroform purification, silica-based methods, and extraction methods based on magnetic glass particle.

Methods used in the present invention are compatible with most, if not all, commercially-available nucleic acid extraction compositions and methods, such as, but not limited to QiaAmp® DNA Mini kit (Qiagen®, Hilden, Germany), MagNA Pure 96 System (Roche Diagnostics, USA), and the NucIiSENS® easyMAG® extraction system (bioMerieux, France).

After nucleic acid extraction, a sample enrichment step (pre-amplification) may performed. The pre-amplification step can be accomplished by any methods know in the art, for example by PCR. Preferable the sample enrichment step is performed using nested PCR which allows for simultaneous amplification of several target genes using multiplex PCR.

After amplification, antibiotic resistance genes are detected by any method known in the art, and preferably by multiplex real time PCR formats such as nanofluidic, microfluidic chip detection real time PCR instrumentation such as Fluidigm Biomark; bead based multiplex detection systems such as Luminex; single target or low multiplex PCR format instrumentation such as Roche Light Cycler; droplet PCR/digital PCR detection system such as Raindances's RainDrop System; or next generation sequencing technology such as Illumina MiSeq, or semiconductor sequencing such as Ion Torrent's, Ion PGM® System.

Whole genome sequencing methods known in the art are particularly suitable for detecting antibiotic resistance genes.

In one embodiment, the present invention provides oligonucleotide primer and probes sequences to specific antibiotic resistance genes. Any primers and probes may be used in the present invention as long as the primers and probes are designed to amplify and detect an antibiotic resistance gene. Additionally, nucleic acid segments, e.g., adapters, may be designed for use in next generation sequencing methods. Methods for designing useful primers, probes, and adapters are well known in the art.

Subsequent to the method steps described herein for determining an appropriate therapeutic regimen for treating an infection caused by antibiotic resistant bacteria, the infection source may be cultured. Culturing the infection source uses methods well-known in the art. Further tests, e.g., antibiotic challenge, PCR genotyping, and whole genome sequencing, may be performed on the cultured bacteria. These further tests supplement and confirm the results obtained from methods previously described herein.

Generation and use of the herein-described databases may be implemented in any of numerous ways. For example, implementations of the subject matter described herein may be realized in digital electronic circuitry, integrated circuitry, specially designed ASICs (application specific integrated circuits), computer hardware, firmware, software, and/or combinations thereof. When implemented in software, the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital assistant (PDA), a smart phone, or any other suitable portable or fixed electronic device.

Also, a computer may have one or more input and output devices. These devices may be used, among other things, to present a user interface. Examples of output devices that may be used to provide a user interface include printers or display screens, such as CRT (cathode ray tube) or LCD (liquid crystal display) monitors, for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible format. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Generation and use of the herein-described databases may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

The herein-described databases and programs for generating same may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. As used herein, “machine-readable medium” refers to any computer program product or apparatus (e.g., a magnetic disc, an optical disk, memory, a Programmable Logic Device (PLD)) used to provide machine instructions and/or data to a programmable processor, including a machine-readable medium that receives machine instructions as a “machine-readable signal,” which includes any signal used to provide machine instructions and/or data to a programmable processor.

Generation and use of the herein-described databases can be implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Additional teaching relevant to the present invention are described in one or both of WO 2015/138991 and WO 2015/184017, each of which is incorporated herein by reference in its entirety.

Table 1, below, associates particular antibiotic resistance genes (or families of genes) with specific antibiotics to which the gene confers resistance.

TABLE 1

| | Antibiotic Resistance Gene |
|---|---|
| Antibiotic Class | Family and Assay Name |
| Aminoglycoside | aac(3)-Ia |
| Aminoglycoside | aac(3)-Ic |
| Aminoglycoside | aac(3)-Id/e |
| Aminoglycoside | aac(3)-II(a-d) |
| Aminoglycoside | aac(3)-IV |
| Aminoglycoside | aac(6′)-Ia |
| Aminoglycoside | aac(6′)-Ib/Ib-cr |
| Aminoglycoside | aac(6′)-Ic |
| Aminoglycoside | aac(6′)-Ie |
| Aminoglycoside | AAC(6′)-IIa |
| Aminoglycoside | aadA12-A24 |
| Aminoglycoside | aadA16 |
| Aminoglycoside | aadA3/A8 |
| Aminoglycoside | aadA5/A5 |
| Aminoglycoside | aadA6/A10/A11 |
| Aminoglycoside | aadA7 |
| Aminoglycoside | aadA9 |
| Aminoglycoside | ANT(2″)-Ia |
| Aminoglycoside | ant(3″)-Ia |
| Aminoglycoside | ant(3″)-II |
| Aminoglycoside | aph(3′)-Ia/c |
| Aminoglycoside | aph(3′)-IIb-A |
| Aminoglycoside | aph(3′)-IIb-B |
| Aminoglycoside | aph(3′)-IIb-C |
| Aminoglycoside | aph(3′)-IIIa |
| Aminoglycoside | aph(3′)-VIa |
| Aminoglycoside | aph(3′)-Vib |
| Aminoglycoside | aph(3′)-XV |
| Aminoglycoside | aph(4)-Ia |
| Aminoglycoside | aph(6)-Ic |
| Aminoglycoside | strA |
| Aminoglycoside | strB |
| AmpC | ACC-1 |
| AmpC | ACC-3 |
| AmpC | ACT-1 |
| AmpC | ACT-5 |
| AmpC | CFE-1 |
| AmpC | CMY-1 |
| AmpC | CMY-2 |
| AmpC | CMY-41 |
| AmpC | CMY-70 |
| AmpC | DHA |
| AmpC | FOX-1 |
| AmpC | GIM-1 |
| AmpC | IMI-1 |
| AmpC | MIR-1 |
| AmpC | MOX-1 |
| AmpC | MOX-5 |
| AmpC | NMC-A |
| Carbapenemase | IMP-1 |
| Carbapenemase | IMP-2 |
| Carbapenemase | KPC-1 |
| Carbapenemase | MCR-1 |
| Carbapenemase | NDM-1 |
| Carbapenemase | OXA-51 |
| Carbapenemase | OXA-23 |
| Carbapenemase | OXA-24 |
| Carbapenemase | OXA-48 |
| Carbapenemase | OXA-54 |
| Carbapenemase | OXA-55 |
| Carbapenemase | OXA-62 |
| Carbapenemase | SFC-1 |
| Carbapenemase | SME-1 |
| Carbapenemase | SPM-1 |
| Carbapenemase | VIM-13 |
| Carbapenemase | VIM-1 |
| Carbapenemase | VIM-2 |
| Carbapenemase | VIM-5 |
| Cephalosporinase | BEL-1 |
| Cephalosporinase | BES-1 |
| Cephalosporinase | CTX-M-1 |
| Cephalosporinase | CTX-M-8/25 |
| Cephalosporinase | CTX-M-2 |
| Cephalosporinase | CTX-M-9 |
| Cephalosporinase | TEM-G238 & E240K |
| Cephalosporinase | GES-1 |
| Cephalosporinase | IMP-5 |
| Cephalosporinase | OXA-10 |
| Cephalosporinase | OXA-18 |
| Cephalosporinase | OXA-2 |
| Cephalosporinase | OXA-45 |
| Cephalosporinase | OXA-50 |
| Cephalosporinase | OXA-58 |
| Cephalosporinase | PER-1 |
| Cephalosporinase | TEM-R164H |
| Cephalosporinase | SHV-G238 & E240K |
| Cephalosporinase | SHV-G238S & E240K |
| Cephalosporinase | SHV-G238S & E240 |
| Cephalosporinase | SHV-G156D |
| Cephalosporinase | SIM-1 |
| Cephalosporinase | TEM-G238S & E240K |
| Cephalosporinase | TEM-E104K |
| Cephalosporinase | TEM-R164C |
| Cephalosporinase | TEM-R164S |
| Cephalosporinase | TEM-G238S & E240 |
| Cephalosporinase | TLA-1 |
| Cephalosporinase | VEB-1 |
| Fluoroquinolone | E. coli GyrA |
| Fluoroquinolone | K. pneumoniae GyrA |
| Fluoroquinolone | E. cloacae GyrA |
| Fluoroquinolone | P. aeruginosa GyrA |
| Fluoroquinolone | E. coli parC |
| Fluoroquinolone | K. pneumoniae parC |
| Fluoroquinolone | E. cloacae parC |
| Fluoroquinolone | P. aeruginosa parC |
| macrolides | ere(A) |
| macrolides | ere(B) |
| macrolides | erm(B) |
| macrolides | mph(A) |
| macrolides | mph(D) |
| macrolides | mph(E) |
| macrolides | msr(E) |
| P. aeruginosa | OXA-50 |
| Penicillinase | OXA-60 |
| Penicillinase | SHV-G238 & E240 (WT) |
| Penicillinase | SHV-G156 (WT) |
| Penicillinase | TEM-E104 (WT) |
| Penicillinase | TEM-R164 (WT) |
| Penicillinase | TEM-G238 & E240 (WT) |
| Quinolone | QnrA1 |
| Quinolone | QnrA3 |
| Quinolone | QnrB10 |
| Quinolone | QnrB11 |
| Quinolone | QnrB13 |
| Quinolone | QnrB1 |
| Quinolone | QnrB31 |
| Quinolone | QnrB21 |
| Quinolone | QnrB22 |
| Quinolone | QnrB27 |
| Quinolone | QnrB2 |
| Quinolone | QnrD1 |
| Quinolone | QnrS1 |
| Quinolone | QnrS2 |
| Quinolone | QnrVC1 |
| Quinolone | QnrVC4 |
| Quinolone Efflux Pump | oqxA |
| Quinolone Efflux Pump | oqxB |
| ribosomal methyl transferase | armA |
| ribosomal methyl transferase | rmtB |
| ribosomal methyl transferase | rmtF |
| sulfonamide | Sul1 |
| sulfonamide | Sul2 |
| sulfonamide | Sul3 |
| tetracycline | tet(A) |
| tetracycline | tetA(B) |
| tetracycline | tetA(G) |
| tetracycline | tetAJ |
| tetracycline | tetG |
| trimethoprim | dfr19/dfrA18 |
| trimethoprim | dfrA12 |
| trimethoprim | dfrA14 |
| trimethoprim | dfrA15 |
| trimethoprim | dfrA16 |
| trimethoprim | dfrA17 |
| trimethoprim | dfrA1 |
| trimethoprim | dfrA23 |
| trimethoprim | dfrA27 |
| trimethoprim | dfrA5 |
| trimethoprim | dfrA7 |
| trimethoprim | dfrA8 |
| trimethoprim | dfrB1/dfr2a |
| trimethoprim | dfrB2 |
| trimethoprim | dhfrB5 |
| Vancomycin | VanA |
| | floR |
| | OXA-1 |
| | OXA-9 |
| | PSE-1 |

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting.

### Examples

### Example 1: Klebsiella and E. coli Sensitivities to a Plurality of Antibiotics

366 bacterial isolates of Klebsiella pneumoniae or Klebsiella oxytoca were collected with known minimal inhibitory concentrations (MIC) for several antibiotics based on phenotypic antibiotic susceptibility testing (AST). The 366 isolates were tested for the presence of several antibiotic resistance genes using polymerase chain reaction (PCR). The 366 Klebsiella isolates were randomly assigned to a training set of 297 isolates and a test set of 69 isolates.

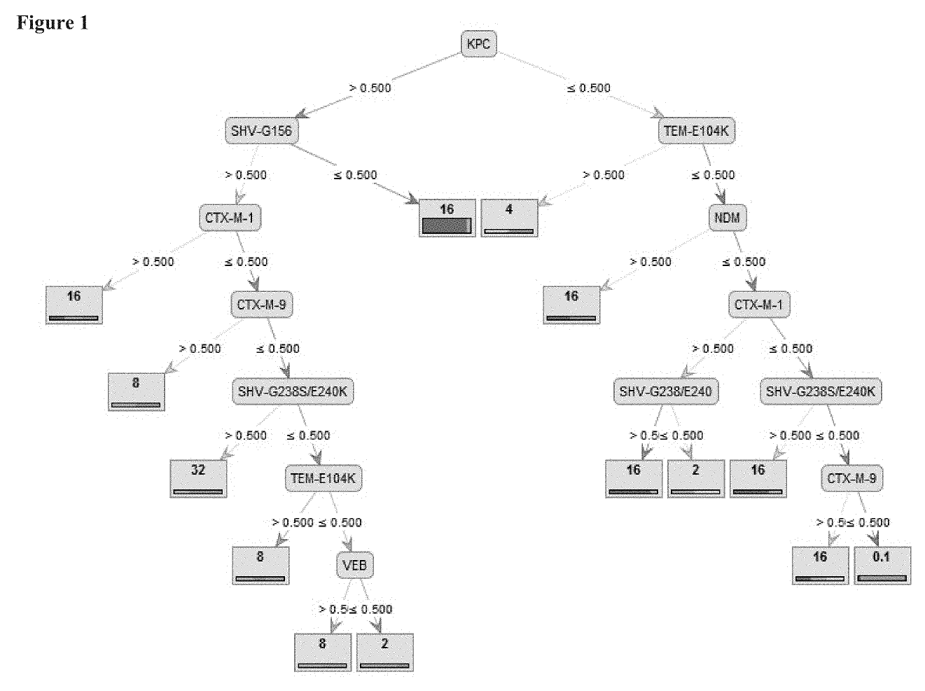

Antibiotic resistance gene results and phenotypic AST results from the training set were combined to create a predictive algorithm for susceptibility to the antibiotic Cefepime using decision tree analysis from the software package RapidMiner Studio (FIG. 1). The decision tree included positive/negative results for the antibiotic resistance genes KPC, CTX-M-1, CTX-M-9, VEB, and NDM. The decision tree also included gene results from wild type versions of the antibiotic resistance genes TEM and SHV plus particular amino acid codon genotypes of TEM and SHV associated with an extended spectrum beta-lactamase (ESBL) phenotype (SHV-G156, SHV-G238S/E240K, TEM-E104K, and SHV-G230/E240).

The decision tree was used to evaluate antibiotic resistance gene results from the test set of sixty nine isolates to predict MIC values that were compared with measured MIC values from phenotypic AST (Table 2). Predicted and measured phenotypic AST results from Table 2 were used to create a 2×2 table based on a Cefepime MIC breakpoint of less than 4 μg/mL for susceptibility and 4 μg/mL or higher for non-susceptibility (Table 3). Gene test results predict phenotypic AST for Cefepime with values of 97% sensitivity, 91% specificity, 98% positive predictive value (PPV) and 83% negative predictive value (NPV) from Table 3.

TABLE 2

| | Number of isolates as | Predicted number of isolates |
|---|---|---|
| Cefepime MIC | measured by phenotypic | based on the presence of |
| (μg/mL) | AST | antibiotic resistance genes |
| 0.1 | 8 | 9 |
| 0.5 | 1 | 0 |
| 1 | 1 | 0 |
| 2 | 1 | 3 |
| 8 | 4 | 1 |
| 16 | 53 | 55 |
| 32 | 1 | 1 |

TABLE 3

| | Non-Susceptible to | Susceptible to |
|---|---|---|
| | Cefepime as measured | Cefepime as measured |
| | by phenotypic AST | by phenotypic AST |
| Resistance Genes Predict | 56 | 1 |
| Non-Susceptible | | |
| Resistance Genes Predict | 2 | 10 |
| Susceptible | | |

Similar analyses were performed with the same set of Klebsiella isolates (Table 4) and a set of Escherichia coli isolates (Table 5) for the antibiotics Ceftazidime, Ertapenem, Meropenem, and Imipenem.

TABLE 4

| | Cef- | | | | |
|---|---|---|---|---|---|
| | tazidime | Cefepime | Ertapenem | Meropenem | Imipenem |
| Sensitivity | 96% | 97% | 98% | 99% | 99% |
| Specificity | 89% | 91% | 93% | 100% | 100% |
| PPV | 99% | 98% | 98% | 100% | 100% |
| NPV | 73% | 83% | 93% | 91% | 50% |

TABLE 5

| | Cef- | | | | |
|---|---|---|---|---|---|
| | tazidime | Cefepime | Ertapenem | Meropenem | Imipenem |
| Sensitivity | 96% | 97% | 98% | 99% | 99% |
| Specificity | 89% | 91% | 93% | 100% | 100% |
| PPV | 99% | 98% | 98% | 100% | 100% |
| NPV | 73% | 83% | 93% | 91% | 50% |

### Example 2: Pseudomonas, E. coli, and K. pneumoniae Sensitivities to a Plurality of Antibiotics

Thirty Pseudomonas aeruginosa isolates with known minimal inhibitory concentrations (MIC) for several antibiotics based on phenotypic antibiotic susceptibility testing (AST) were collected. Whole genome sequencing was used to obtain genotypes for amino acid codons 83 and 87 of the gyraseA gene, amino acid codon 80 of the parC gene, and amino acid codon 475 of the parE gene (Table 6).

TABLE 6

| | Measured | Predicted | | | | | | | | | |
|---|---|---|---|---|---|---|---|---|---|---|---|
| | Levofloxacin MIC | Levofloxacin MIC | Amino Acid (1 = positve, 0 = negative) | | | | | | | | |
| | (ug/mL) from | (ug/mL) based on | gyrA | gyrA | gyrA | gyrA | gyrA | parC | parC | parC | parE |
| Isolate | phenotypic AST | genotypes | 83I | 83T | 87D | 87N | 87Y | 80L | 80S | 80W | 475D |
| 1 | 8 | 8 | 1 | 0 | 1 | 0 | 0 | 1 | 0 | 0 | 1 |
| 2 | 8 | 8 | 1 | 0 | 0 | 1 | 0 | 1 | 0 | 0 | 1 |
| 3 | 8 | 8 | 1 | 0 | 1 | 0 | 0 | 0 | 0 | 1 | 1 |
| 4 | 8 | 8 | 1 | 0 | 1 | 0 | 0 | 1 | 0 | 0 | 1 |
| 5 | 8 | 8 | 1 | 0 | 1 | 0 | 0 | 0 | 1 | 0 | 1 |
| 6 | 8 | 8 | 1 | 0 | 1 | 0 | 0 | 1 | 0 | 0 | 1 |
| 7 | 8 | 8 | 1 | 0 | 1 | 0 | 0 | 1 | 0 | 0 | 1 |
| 8 | 8 | 8 | 1 | 0 | 1 | 0 | 0 | 1 | 0 | 0 | 1 |
| 9 | 8 | 8 | 1 | 0 | 1 | 0 | 0 | 0 | 1 | 0 | 1 |
| 10 | 8 | 8 | 1 | 0 | 1 | 0 | 0 | 1 | 0 | 0 | 1 |
| 11 | 4 | 8 | 1 | 0 | 1 | 0 | 0 | 1 | 0 | 0 | 1 |
| 12 | 4 | 8 | 1 | 0 | 1 | 0 | 0 | 1 | 0 | 0 | 1 |
| 13 | 4 | 8 | 1 | 0 | 1 | 0 | 0 | 1 | 0 | 0 | 1 |
| 14 | 4 | 4 | 1 | 0 | 0 | 0 | 1 | 1 | 0 | 0 | 1 |
| 15 | 4 | 8 | 1 | 0 | 1 | 0 | 0 | 0 | 1 | 0 | 1 |
| 16 | 4 | 8 | 1 | 0 | 1 | 0 | 0 | 1 | 0 | 0 | 1 |
| 17 | 4 | 0.5 | 0 | 1 | 1 | 0 | 0 | 0 | 1 | 0 | 1 |
| 18 | 4 | 0.5 | 0 | 1 | 1 | 0 | 0 | 0 | 1 | 0 | 1 |
| 19 | 4 | 0.5 | 0 | 1 | 1 | 0 | 0 | 0 | 1 | 0 | 1 |
| 20 | 4 | 0.5 | 0 | 1 | 1 | 0 | 0 | 0 | 1 | 0 | 1 |
| 21 | 1 | 8 | 1 | 0 | 1 | 0 | 0 | 0 | 1 | 0 | 1 |
| 22 | 1 | 0.5 | 0 | 1 | 1 | 0 | 0 | 0 | 1 | 0 | 1 |
| 23 | 0.5 | 0.5 | 0 | 1 | 1 | 0 | 0 | 0 | 1 | 0 | 1 |
| 24 | 0.5 | 0.5 | 0 | 1 | 1 | 0 | 0 | 0 | 1 | 0 | 1 |
| 25 | 0.5 | 0.5 | 0 | 1 | 1 | 0 | 0 | 0 | 1 | 0 | 1 |
| 26 | 0.5 | 0.5 | 0 | 1 | 1 | 0 | 0 | 0 | 1 | 0 | 1 |
| 27 | 0.5 | 0.5 | 0 | 1 | 1 | 0 | 0 | 0 | 1 | 0 | 1 |
| 28 | 0.5 | 0.5 | 0 | 1 | 1 | 0 | 0 | 0 | 1 | 0 | 1 |
| 29 | 0.5 | 0.5 | 0 | 1 | 1 | 0 | 0 | 0 | 1 | 0 | 1 |
| 30 | 0.25 | 0.5 | 0 | 1 | 1 | 0 | 0 | 0 | 1 | 0 | 1 |

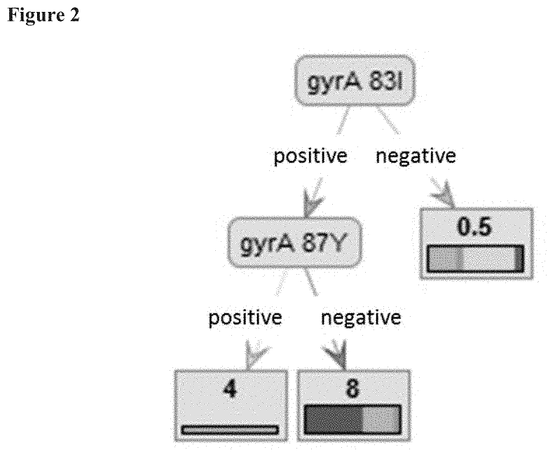

Genotype results for the three genes and phenotypic AST results for the antibiotic Levofloxacin were analyzed using decision tree analysis from the software package RapidMiner Studio (FIG. 2) to predict Levofloxacin MIC values based on genotypes for the three genes (Table 6). A 2×2 table, as shown in Table 7, was created using measured phenotypic AST results for Levofloxacin and predicted Levofloxacin MIC values from genotypes for the three genes based on a Levofloxacin MIC breakpoint of less than 4 μg/mL for susceptibility and 4 μg/mL or higher for non-susceptibility. Genotypes predict phenotypic AST for Levofloxacin with values of 80% sensitivity, 90% specificity, 94% positive predictive value (PPV) and 69% negative predictive value (NPV) from Table 7.

TABLE 7

| | Non-Susceptible | Susceptible to |
|---|---|---|
| | to Levofloxacin | Levofloxacin as |
| | as measured by | measured by |
| | phenotypic AST | phenotypic AST |
| Genotypes Predict Non- | 16 | 1 |
| Susceptible | | |
| Genotypes Predict | 4 | 9 |
| Susceptible | | |

Similar analyses were performed for E. coli and K. pneumoniae with the antibiotics Levofloxacin and Ciprofloxacin as summarized in Table 8.

TABLE 8

| | Pseudomonas aeruginosa | | Escherichia coli | | Klebsiella pneumoniae | |
|---|---|---|---|---|---|---|
| | Levofloxacin | Ciprofloxacin | Levofloxacin | Ciprofloxacin | Levofloxacin | Ciprofloxacin |
| Sensitivity | 80% | | 100% | 100% | 100% | 100% |
| Specificity | 90% | | 100% | 100% | 100% | 100% |
| PPV | 94% | | 100% | 100% | 100% | 100% |
| NPV | 69% | | 100% | 100% | 100% | 100% |

### Example 3: Predicting Antibiotic Resistance in E. coli from Resistance Genes

1496 clinical isolates of E. coli were genotyped for several antibiotic resistant genes, and statistical methods were used to predict phenotypic antibiotic resistance from resistance genes. Resistance genes predicted phenotypic antibiotic susceptibility test results for 25 antibiotics including penicillins, cephalosporins, carbapenems, aminoglycosides, fluoroquinolones, tetracyclines and trimethoprim/sulfamethoxazole with 75 to 98% accuracy across the antibiotics.

Phenotypic antibiotic susceptibility testing was performed and an antibiotic response of resistant, intermediate or susceptible was assigned to each E. coli isolate per antibiotic based on minimal inhibitory concentrations as described in the MicroScan product insert. Phenotypic antibiotic susceptibility testing was performed on the 1496 E. coli isolates using the MicroScan WalkAway plus System and the Neg MIC 45 panel (P/N B1017-424) which covers 25 antibiotics. Cryopreserved isolates were sub-cultured twice on blood agar plates prior to antibiotic susceptibility testing. The MicroScan instrument was used to assign an antibiotic response of resistant, intermediate or susceptible for each isolate per antibiotic based on minimal inhibitory concentrations as described in the MicroScan product insert. Assignments of resistant or intermediate were combined and reported as resistant in this example. Assignments of susceptible are reported as such in this example.

Polymerase chain reaction (PCR) was used to evaluate the 1496 E. coli isolates for antibiotic resistance genes coding penicillinases, cephalosporinases, carbapenemases, AmpC beta-lactamases, aminoglycoside modifying enzymes, ribosomal methyltransferases, dihydrofolate reductase, plasmid-mediated quinolone resistance, macrolide modifying enzymes, sulfonamide resistance, plasmid-mediated pumps and tetracycline/macrolide efflux.

For PCR, 0.5 McFarland standards were prepared using single colonies of E. coli obtained from the same blood agar plates used for antibiotic susceptibility testing. Total nucleic acids were extracted from 500 μL of each McFarland standard using the Roche MagNA Pure 96 DNA and Viral NA Large Volume Kit (P/N 06374891001) on the MagNA Pure 96 System. PCR was performed using primers and fluorescent reporter probes (Applied Biosystems Custom TaqMan® MGB™ Probes with 5′-FAM™ or 5′-VIC™ with a 3′ non-fluorescent quencher). All PCRs used dUTP instead of TTP along with uracil-DNA glycosylase prior to guard against accidental amplicon contamination. An internal amplification control (gBlocks Gene Fragment from Integrated DNA Technologies) was prepared in 1 μg/mL of calf thymus DNA in TRIS-EDTA, pH 8 (Fisher catalog # BP2473-1) and added to all samples to monitor potential PCR inhibition. gBlocks covering all target amplicon sequences were used as positive PCR control samples.

PCR was performed with Fluidigm's BioMark HD System using 96.96 Dynamic Array™ IFC Arrays, a microfluidic system capable of analyzing 96 samples with 96 separate PCR assays. Each PCR contained 3 nL of extracted DNA plus 610 nmol/L each PCR primer, 340 nmol/L fluorescent reporter probe, and 0.91× ThermoFisher TaqPath qPCR MasterMix, CG (P/N A16245). Most assays were two-plex PCRs containing two primers and a FAM probe for one target plus two primers and a VIC probe for the other target. PCR was performed with the following cycling program 2 min at 50° C., 10 minutes at 95° C. and 40 cycles of 15 seconds at 95° C., 1 minute at 60° C.

General linear models were used to predict phenotypic resistance from resistance genes across the 1496 E. coli isolates. Models were generated for each antibiotic and evaluated for accuracy through a series of stepwise gene additions/eliminations and 10-fold cross validation repeated three times. Final models were chosen based on highest cross-validation accuracy and smallest accuracy variance.

Prediction of phenotypic resistance from resistance genes for each antibiotic across the 1496 E. coli isolates is summarized (Table 9) in terms of accuracy, Kappa, sensitivity, specificity, positive predictive values (PPV), negative predictive values (NPV) and area under the curve (AUC) for Receiver Operator Curves (ROC). The 1496 E. coli isolates exhibited balanced distribution of measured phenotypic resistance and susceptibility for several antibiotics allowing strong prediction of phenotypic antibiotic resistance from PCR results (accuracy, Kappa) for ciprofloxacin (98%, 0.94), levofloxacin (98%, 0.95), tetracycline (96%, 0.91), gentamycin (96%, 0.91), trimethoprim/sulfamethoxazole (94%, 0.88) and tobramycin (94%, 0.87). Weaker predictive models were obtained (accuracy, Kappa) for ampicillin/sulbactam (89%, 0.58), piperacillin/tazobactam (85%, 0.27), cefoxitin (83%, 0.36), amoxicillin/clavulanate (80%, 0.59) and ticarcillin/clavulanate (75%, 0.48).

Modeled PCR results (Table 9) accurately predicted phenotypic antibiotic resistance (accuracy, Kappa) for ceftazidime (96%, 0.79), ceftriaxone (96%, 0.78), cefotaxime (96%, 0.75), cefuroxime (96%, 0.72), cefepime (95%, 0.76) and aztreonam (95%, 0.71), although the statistical significance of these predictions was limited by imbalanced distribution of measured phenotypic resistance and susceptibility for these antibiotics across the 1496 E. coli isolates.

The E. coli isolates exhibited even more pronounced imbalance of susceptible and resistant phenotypes for cefazolin, ampicillin, piperacillin, ertapenem, meropenem, imipenem, amikacin and tigecycline, which limited statistical prediction of antibiotic resistance for these antibiotics (Table 9). For example, the genotype-based models predicted antibiotic resistance for cefazolin, ampicillin and piperacillin with high accuracy and sensitivity but low Kappa values, in part because the vast majority of isolates exhibited phenotypic resistance to these antibiotics (Table 9). In contrast, the PCR models predicted antibiotic resistance with low sensitivity and Kappa values for ertapenem, meropenem, imipenem, amikacin and tigecycline. Predictive resistance genes could not be identified for these antibiotics with high statistical power, in part because the vast majority of isolates exhibited phenotypic susceptibility to these antibiotics even though many of the resistant isolates were positive for resistance genes associated with carbapenems, aminoglycosides and macrolides.

Predictions of antibiotic resistance from resistance genes were also tabulated in terms of true/false positives and negatives for the 1496 E. coli isolates across ciprofloxacin, levofloxacin, tetracycline, gentamycin, trimethoprim/sulfamethoxazole, tobramycin, ampicillin/sulbactam, piperacillin/tazobactam, cefoxitin, amoxicillin/clavulanate, ticarcillin/clavulanate, ceftazidime, ceftriaxone, cefotaxime, cefuroxime, cefepime and aztreonam in Table 10.

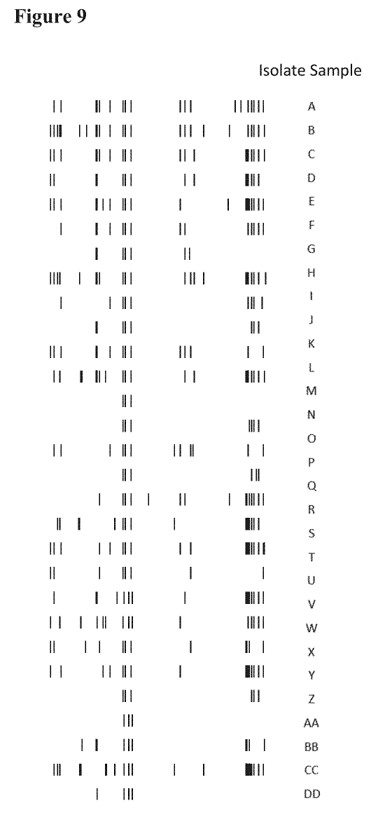

High resolution analysis of antibiotic resistance genes can provide strain type information for highly resistant strains. Individual heat maps resembling barcodes for 30 of the 1496 E. coli isolates chosen at random are provided here as an illustration (FIG. 9). Antibiotic resistance genes are ordered horizontally along the heat maps with the presence of resistance genes indicated by a black bar. The individual heat maps are different because each of the 30 isolates has a unique combination of antibiotic resistance genes, suggesting the isolates are different strains of E. coli. It should be noted that identical heat maps do not necessarily indicate identical strains especially for less resistant isolates.

TABLE 9
Prediction of antibiotic resistance from resistance genes across the 1496 E. coli isolates

| | Measured Phenotype | | | Predicted Phenotype from Resistance Genes | | | | | |
|---|---|---|---|---|---|---|---|---|---|
| Antibiotic | Susceptible (%) | Resistant (%) | Accuracy (%) | Kappa | Sensitivity (%) | Specificity (%) | PPV (%) | NPV (%) | AUC |
| Levofloxacin | 19 | 81 | 98 | 0.95 | 99 | 96 | 99 | 96 | 0.98 |
| Ciprofloxacin | 18 | 82 | 98 | 0.94 | 98 | 97 | 99 | 93 | 0.98 |
| Gentamicin | 61 | 39 | 96 | 0.91 | 94 | 97 | 95 | 96 | 0.98 |
| Tetracycline | 32 | 68 | 96 | 0.91 | 96 | 95 | 98 | 92 | 0.97 |
| Trimethoprim/ | 36 | 64 | 94 | 0.88 | 96 | 91 | 95 | 93 | 0.96 |
| Sulfamethoxazole | | | | | | | | | |
| Tobramycin | 45 | 55 | 94 | 0.87 | 94 | 93 | 94 | 93 | 0.96 |
| Ceftazidime | 9 | 91 | 96 | 0.79 | 98 | 85 | 99 | 78 | 0.94 |
| Ceftriaxone | 8 | 92 | 96 | 0.78 | 97 | 89 | 99 | 72 | 0.96 |
| Cefepime | 10 | 90 | 95 | 0.76 | 96 | 86 | 98 | 73 | 0.95 |
| Cefotaxime | 9 | 91 | 96 | 0.75 | 97 | 82 | 98 | 73 | 0.95 |
| Cefuroxime | 7 | 93 | 96 | 0.72 | 97 | 85 | 99 | 65 | 0.95 |
| Aztreonam | 9 | 91 | 95 | 0.71 | 96 | 81 | 98 | 68 | 0.94 |
| Amoxicillin/ | 43 | 57 | 80 | 0.6 | 80 | 80 | 84 | 75 | 0.88 |
| K Clavulanate | | | | | | | | | |
| Ampicillin/ | 16 | 84 | 89 | 0.58 | 94 | 63 | 93 | 66 | 0.89 |
| Sulbactam | | | | | | | | | |
| Ticarcillin/ | 56 | 44 | 75 | 0.49 | 74 | 76 | 70 | 79 | 0.81 |
| K Clavulanate | | | | | | | | | |
| Ertapenem | 98 | 4 | 97 | 0.49 | 35 | 100 | 85 | 97 | 0.84 |
| Meropenem | 98 | 2 | 98 | 0.46 | 38 | 100 | 61 | 99 | 0.91 |
| Cefazolin | 5 | 95 | 95 | 0.43 | 98 | 41 | 97 | 53 | 0.93 |
| Cefoxitin | 79 | 21 | 83 | 0.35 | 30 | 97 | 75 | 84 | 0.76 |
| Piperacillin/ | 84 | 16 | 85 | 0.31 | 28 | 96 | 60 | 87 | 0.84 |
| Tazobactam | | | | | | | | | |
| Amikacin | 94 | 6 | 94 | 0.22 | 14 | 100 | 72 | 95 | 0.89 |
| Ampicillin | 3 | 97 | 97 | 0.04 | 100 | 2 | 97 | 25 | 0.91 |
| Imipenem | 97 | 3 | 97 | 0 | 0 | 100 | | 97 | 0.7 |
| Piperacillin | 4 | 96 | 96 | 0 | 100 | 0 | 96 | | 0.92 |
| Tigecycline | 99.6 | 0.4 | 100 | 0 | 0 | 100 | | 100 | 0.79 |

TABLE 10
Predictions of antibiotic resistance from resistance genes in terms of true/false
positives and negatives for the 1496 E. coli isolates

| | | Prediction of Antibiotic Resistance from Resistance Genes | | | |
|---|---|---|---|---|---|
| Antibiotic Class | Antibiotic | True Positives | True Negatives | False Positives | False Negatives |
| Fluoroquinolones | Levofloxacin | 1168 | 279 | 7 | 42 |
| | Ciprofloxacin | 1200 | 268 | 7 | 20 |
| Aminoglycosides | Gentamycin | 545 | 887 | 26 | 37 |
| | Tobramycin | 770 | 631 | 47 | 47 |
| Macrolide | Tetracycline | 986 | 449 | 23 | 37 |
| | Trimethoprim/Sulfamethoxazole | 916 | 495 | 46 | 39 |
| Penicillin/Beta- | Amoxicillin/K Clavulanate | 687 | 513 | 127 | 168 |
| lactamase Inhibitor | Ampicillin/Sulbactam | 1174 | 152 | 90 | 79 |
| | Ticarcillin/K Clavulanate | 487 | 632 | 205 | 172 |
| | Pipercillin/Tazobactam | 69 | 1206 | 46 | 174 |
| Cephalosporins | Cefepime | 1291 | 132 | 22 | 50 |
| | Cefotaxime | 1325 | 107 | 24 | 39 |
| | Ceftazidime | 1329 | 114 | 20 | 33 |
| | Ceftriaxone | 1333 | 107 | 13 | 42 |
| | Cefuroxime | 1348 | 86 | 15 | 46 |
| Monobactam | Aztreonam | 1302 | 113 | 26 | 54 |

1. A method for predicting phenotypic antibiotic resistance of a pathogenic bacteria comprising:
a. detecting in the bacteria the presence or absence of at least one antibiotic resistance gene to produce an infection source profile; and
b. comparing the infection source profile to a control profile thereby predicting the phenotypic antibiotic resistance of the bacteria.

2. The method of claim 1, wherein the pathogenic bacteria is obtained from a biological sample from a subject having or suspected of having a pathogenic bacterial infection or is collected from the environment.

3. (canceled)

4. The method of claim 1, further comprising making a contact precautions recommendation.

5. The method of claim 4, wherein the contact precautions recommendation includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

6. A method for determining the minimal inhibitory concentration (MIC) of an antibiotic for treatment of a bacterial infection in a subject comprising:
a. obtaining a biological sample from the subject;
b. detecting in the biological sample the presence or absence of at least one antibiotic resistance gene to produce an infection source profile; and
c. comparing the infection source profile to a control profile thereby identifying the MIC of the antibiotic for treatment of the bacterial infection.

7. The method of claim 6, further comprising choosing and administering the antibiotic to the subject at a dose based on the MIC.

8. The method of claim 6, wherein the subject has or is suspected of having a bacterial infection.

9. The method of claim 6, wherein the biological sample comprises pathogenic bacteria.

10. The method of claim 1, wherein the pathogenic bacteria is Escherichia coli, Klebsiella pneumoniae, Enterobacter cloacae, Pseudomonas aeruginosa, Proteus mirabilis, Klebsiella oxytoca, Streptococcus pneumoniae, Staphylococcus aureus, Streptococcus anginosus, Streptococcus constellatus, Streptococcus salivarius, Enterobacter aerogenes, Serratia marcescens, Acinetobacter baumannii, Citrobacter freundii, Morganella morganii, Legionella pneumophila, Moraxella catarrhalis, Haemophilus influenzae, Haemophilus parainfluenzae, Mycoplasma pneumoniae, Chlamydophila pneumoniae, Clostridium species, or Bacteroides fragilis.

11. The method of claim 1, wherein the antibiotic resistance gene is aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, or VIM-5.

12. The method of claim 1, wherein the antibiotic is Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, or Vancomycin.

13. The method of claim 1, wherein the control profile is a database.

14. The method of claim 1, wherein the biological sample is an anal swab, a rectal swab, a skin swab, a nasal swab, a wound swab, stool, blood, plasma, serum, urine, sputum, respiratory lavage, cerebrospinal fluid, or a bacterial culture.

15. A method for determining the minimal inhibitory concentration (MIC) of an antibiotic for a bacterial isolate:
a. detecting in the bacterial isolate the presence or absence of at least one antibiotic resistance gene to produce an infection source profile; and
b. comparing the infection source profile to a control profile thereby identifying the MIC of the antibiotic for the bacterial isolate.

16. The method of claim 15, wherein the bacterial isolate is obtained from a subject having or suspected of having a bacterial infection or is collected from the environment.

17. (canceled)

18. The method of claim 15, further comprising making a contact precautions recommendation.

19. The method of claim 18 wherein the contact precautions recommendation includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

20. The method of claim 15, wherein the bacterial isolate is from the species Escherichia coli, Klebsiella pneumoniae, Enterobacter cloacae, Pseudomonas aeruginosa, Proteus mirabilis, Klebsiella oxytoca, Streptococcus pneumoniae, Staphylococcus aureus, Streptococcus anginosus, Streptococcus constellatus, Streptococcus salivarius, Enterobacter aerogenes, Serratia marcescens, Acinetobacter baumannii, Citrobacter freundii, Morganella morganii, Legionella pneumophila, Moraxella catarrhalis, Haemophilus influenzae, Haemophilus parainfluenzae, Mycoplasma pneumoniae, Chlamydophila pneumoniae, Clostridium species, or Bacteroides fragilis.

21. The method of claim 15, wherein the antibiotic resistance gene is aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, or VIM-5.

22. The method of claim 15, wherein the antibiotic is Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, or Vancomycin.

23. A method for determining whether an infection source will be susceptible to an antibiotic comprising:
a. obtaining a sample comprising the infection source;
b. detecting in the sample the presence or absence of an antibiotic resistance gene thereby determining an infection source profile; and
c. comparing the infection source profile to a control profile thereby determining whether an infection source will be susceptible to an antibiotic.

24. The method of claim 23, wherein the sample is obtained from a subject having or suspected of having a bacterial infection or is collected from the environment.

25. (canceled)

26. The method of claim 23, further comprising making a contact precautions recommendation.

27. The method of claim 26, wherein the contact precautions recommendation includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

28. A method for generating a database that correlates a genetic profile with a minimal inhibitory concentration (MIC) of an antibiotic comprising:
a. obtaining a plurality of bacterial isolates of a bacterial species or a bacterial strain wherein the MIC of the antibiotic for each bacterial isolate in the plurality is known;
b. determining a genetic profile for each bacterial isolate, wherein the genetic profile comprises the presence or absence of one or more antibiotic resistance genes; and
c. associating each genetic profile for each isolate with its known MIC of the antibiotic, thereby generating a database that correlates a genetic profile with a MIC of the antibiotic.

29. A method for generating a database that correlates a genetic profile with susceptibility to an antibiotic comprising
a. obtaining a plurality of bacterial isolates of a bacterial species or a bacterial strain wherein each bacterial isolate in the plurality has a known susceptibility to at least one antibiotic;
b. determining a genetic profile for each isolate wherein the genetic profile comprises the presence or absence of one or more antibiotic resistance genes; and
c. associating each genetic profile for each isolate with its known susceptibility to the at least one antibiotic, thereby generating a database that correlates a genetic profile with susceptibility to at least one antibiotic.

30. The method of claim 28, wherein the bacterial isolates are selected from the group consisting of Escherichia coli, Klebsiella pneumoniae, Enterobacter cloacae, Pseudomonas aeruginosa, Proteus mirabilis, Klebsiella oxytoca, Streptococcus pneumoniae, Staphylococcus aureus, Streptococcus anginosus, Streptococcus constellatus, Streptococcus salivarius, Enterobacter aerogenes, Serratia marcescens, Acinetobacter baumannii, Citrobacter freundii, Morganella morganii, Legionella pneumophila, Moraxella catarrhalis, Haemophilus influenzae, Haemophilus parainfluenzae, Mycoplasma pneumoniae, Chlamydophila pneumoniae, Clostridium species, and Bacteroides fragilis.

31. The method of claim 28, wherein the antibiotic resistance gene is aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, or VIM-5.

32. The method of claim 28, wherein the antibiotic is Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, or Vancomycin.

33. A database generated by the method of claim 28.

34. A non-transient computer readable medium containing the database of claim 33.

35. A method for predicting phenotypic antibiotic resistance of a pathogenic bacteria comprising:
a. detecting in the bacteria the presence or absence of at least one antibiotic resistance gene to produce an infection source profile; and
b. comparing the infection source profile to the database of claim 33 to predict the phenotypic antibiotic resistance of the bacteria.

36. The method of claim 35, wherein the pathogenic bacteria is obtained from a subject having or suspected of having a pathogenic bacterial infection or is collected from the environment.

37. (canceled)

38. The method of claim 35, further comprising making a contact precautions recommendation.

39. The method of claim 38, wherein the contact precautions recommendation includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

40. A method of identifying the bacterial species or bacterial strain in a sample comprising:
a. detecting in the sample the presence or absence of at least one antibiotic resistance gene to produce a sample profile; and
b. comparing the sample profile to a control profile thereby identifying the bacterial strain in a sample.

41. The method of claim 40, wherein the sample is obtained from a subject having or suspected of having a bacterial infection or is collected from the environment.

42. (canceled)

43. The method of claim 40, further comprising making a contact precautions recommendation.

44. The method of claim 43, wherein the contact precautions recommendation includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

45. A method for predicting phenotypic antibiotic resistance of a pathogenic bacteria comprising:
a. assessing the expression of a plurality of antibiotic resistance genes in the bacteria; and
b. calculating a score from the expression the antibiotic resistance genes wherein the score indicates the phenotypic resistance of the bacteria.

46. The method of claim 45, wherein the bacteria is obtained from a subject having or suspected of having a bacterial infection or is collected from the environment.

47. (canceled)

48. The method of claim 45, further comprising making a contact precautions recommendation.

49. The method of claim 48, wherein the contact precautions includes one or more of the following: isolating the patient to a quarantine area or ward, providing a private room for said patient, donning personal protective apparel upon entering the patient's room, limiting patient mobility, limiting or restricting access of non-colonized or non-infected patients or medical personnel to the patient, or providing dedicated patient care equipment.

50. The method of claim 45, wherein the antibiotic resistance gene is aac(3)-Ia, aac(3)-Ic, aac(3)-Id/e, aac(3)-II(a-d), aac(3)-IV, aac(6′)-Ia, aac(6′)-Ib/Ib-cr, aac(6′)-Ic, aac(6′)-Ie, AAC(6′)-IIa, aadA12-A24, aadA16, aadA3/A8, aadA5/A5, aadA6/A10/A11, aadA7, aadA9, ACC-1, ACC-3, ACT-1, ACT-5, ANT(2″)-Ia, ant(3″)-Ia, ant(3″)-II, aph(3′)-Ia/c, aph(3′)-IIb-A, aph(3′)-IIb-B, aph(3′)-IIb-C, aph(3′)-IIIa, aph(3′)-VIa, aph(3′)-Vib, aph(3′)-XV, aph(4)-Ia, aph(6)-Ic, armA, BEL-1, BES-1, CFE-1, CMY-1, CMY-2, CMY-41, CMY-70, CTX-M-1, CTX-M-2, CTX-M-8/25, CTX-M-9, dfr19/dfrA18, dfrA1, dfrA12, dfrA14, dfrA15, dfrA16, dfrA17, dfrA23, dfrA27, dfrA5, dfrA7, dfrA8, dfrB1/dfr2a, dfrB2, DHA, dhfrB5, E. cloacae GyrA, E. cloacae parC, E. coli GyrA, E. coli parC, ere(A), ere(B), erm(B), floR, FOX-1, GES-1, GIM-1, IMI-1, IMP-1, IMP-2, IMP-5, K. pneumoniae GyrA, K. pneumoniae parC, KPC-1, MCR-1, MIR-1, MOX-1, MOX-5, mph(A), mph(D), mph(E), msr(E), NDM-1, NMC-A, oqxA, oqxB, OXA-1, OXA-10, OXA-18, OXA-2, OXA-23, OXA-24, OXA-45, OXA-48, OXA-50, OXA-50, OXA-51, OXA-54, OXA-55, OXA-58, OXA-60, OXA-62, OXA-9, P. aeruginosa GyrA, P. aeruginosa parC, PER-1, PSE-1, QnrA1, QnrA3, QnrB1, QnrB10, QnrB11, QnrB13, QnrB2, QnrB21, QnrB22, QnrB27, QnrB31, QnrD1, QnrS1, QnrS2, QnrVC1, QnrVC4, rmtB, rmtF, SFC-1, SHV-G238S & E240, SHV-G156 (WT), SHV-G156D, SHV-G238 & E240 (WT), SHV-G238 & E240K, SHV-G238S & E240K, SIM-1, SME-1, SPM-1, strA, strB, Sul1, Sul2, Sul3, TEM-E104 (WT), TEM-E104K, TEM-G238 & E240 (WT), TEM-G238 & E240K, TEM-G238S & E240, TEM-G238S & E240K, TEM-R164 (WT), TEM-R164C, TEM-R164H, TEM-R164S, tet(A), tetA(B), tetA(G), tetAJ, tetG, TLA-1, VanA, VEB-1, VIM-1, VIM-13, VIM-2, or VIM-5.

51. The method of claim 45, wherein the antibiotic is Amikacin, Amoxicillin/K Clavulanate, Ampicillin, Ampicillin/Sulbactam, Aztreonam, Cefazolin, Cefepime, Cefotaxime, Cefotaxime, Cefotaxime/K Clavulanate, Cefoxitin, Ceftazidime, Ceftazidime/K Clavulanate, Ceftriaxone, Cefuroxime, Ciprofloxacin, Ertapenem, Gentamicin, Imipenem, Levofloxacin, Meropenem, Nitrofurantoin, Piperacillin, Piperacillin/Tazobactam, Tetracycline, Ticarcillin/K Clavulanate, Tigecycline, Tobramycin, Trimethoprim/Sulfamethoxazole, Zerbaxa (ceftolozane and tazobactam), imipenem/cilastatin/relebactam, Amoxicillin/K Clavulanate, Ampicillin, Ampicillin/Sulbactam, Cefazolin, Ceftriaxone, Chloramphenicol, Clindamycin, Daptomycin, Erythromycin, Gentamicin, Gentamicin Synergy Screen, Imipenem, Levofloxacin, Linezolid, Meropenem, Moxifloxacin, Nitrofurantoin, Oxacillin, Penicillin, Rifampin, Streptomycin, Synercid, Tetracycline, Trimethoprim/Sulfamethoxazole, or Vancomycin.
